(19) United States
(12) Patent Application Publication
(10) Pub. No.: US 2005/0197684 A1
(43) Pub. Date: Sep. 8, 2005

(54) Process and device for body climate control

(75) Inventor: Jochim Koch, Ratzeburg (DE)

Correspondence Address: MCGLEW & TUTTLE, PC, P.O. BOX 9227, SCARBOROUGH STATION, SCARBOROUGH, NY, 10510-9227, US

(73) Assignee: Drager Safety AG & Co., Lubeck (DE)

(21) Appl. No.: 10/979,713

(22) Filed: Nov. 2, 2004

(30) Foreign Application Priority Data
Mar. 8, 2004 (DE) 10 2004 011 139.1

(51) Int. Cl. A61F 7/00

(52) U.S. Cl. 607/104; 607/108

(57) ABSTRACT

A piece of clothing is designed especially as a vest (4) with ventilation channels (2) which are connected at their inlet and outlet with the environment. At least one temperature sensor (11, 12; 17) determines the body temperature of the user (1) of the piece of clothing. A blower (5) admits ambient air (7) into the ventilation channels (2), and a measuring and evaluating unit (13), is connected with the at least one temperature sensor (11, 12) and the blower (5).

### CROSS REFERENCE TO RELATED APPLICATIONS

This application claims the benefit of priority under 35 U.S.C. § 119 of German application number 10 2004 011 139.1 filed Mar. 8, 2004, the entire contents of which are incorporated herein by reference.

### FIELD OF THE INVENTION

The present invention pertains to a process and device for body climate control, and in particular to clothing with ventilation channels, temperature sensors and a fan, as well as a method of operating the clothing.

### BACKGROUND OF THE INVENTION

The maintenance of a physiologically suitable individual body climate is important for the well-being of humans, but the climate control has hitherto been performed, for example, in air-conditioned rooms without a direct feedback between the well-being of a certain individual and the air conditioner in the room or in the building. In incubators as quasi locally stationary devices, air conditioning for an incubator interior space with feedback to the body temperature of the patient is known in the field of medicine, as is apparent from, e.g., U.S. Pat. No. 5,385,529.

The special problem faced for a long time in firefighting and rescue operations is that persons who are subject to a high physical stress are not sufficiently able to release the body heat generated to the environment. According to U.S. Pat. No. 5,635,909, alarm signals are triggered when preset temperature limit values are exceeded in the protective clothing of a fireman. The fireman, wearing special clothing, is protected from intense external mechanical and thermal effects. On the other hand, this protection from external effects insulates him thermally so intensely that his body temperature can increase very greatly with increasing action/operating time and reach hazardous values. It is known from studies that a considerable percentage, about 40%, of firemen, who die in action, die from cardiovascular disturbances. These cases are clearly linked with the physiological stress and the rise in the body temperature.

Various textiles, which release moisture generated by sweating to the environment by vapor diffusion, have been known from the area of sports and recreation. On the other hand, textiles that offer protection from high external thermal effects are known, so that addition of the properties is to be achieved by the combination of the different materials. However, it was found that the prior-art measures are not sufficient to prevent a rise in the body temperature, for example, in firemen during active use. Even though active cooling suits with ice coolers or vests with active ventilation have become known as well, these concepts do not take into account the fact that the human body has a thermal regulation of its own, and they deliver only a permanent and constant cooling output, without taking into account the individual thermal well-being of the particular person.

### SUMMARY OF THE INVENTION

The object of the present invention is to provide a process and a device for body climate control, so that the temperature of the personal protective clothing is set as a function of individual measured temperature values.

This object is accomplished with a piece of clothing having ventilation channels a temperature sensor determining the temperature near the core of the body, a temperature sensor determining the peripheral temperature of the body, a blower ventilating the ventilation channels with ambient air and with a measuring and evaluating unit connected with the temperature sensors and with the blower. The piece of clothing with the ventilation channels is connected to the environment at their inlet and outlet. The blower is switched on with a preset speed, and the blower is switched off when the measured temperature near the core of the body falls below a limit value. The blower is also switched off when the difference between the measured temperature near the core of the body and the peripheral temperature of the body exceeds a preset limit value. The speed of the blower is otherwise set as a function of the measured temperature near the core of the body and/or the peripheral temperature of the body.

An essential advantage of the present invention is the possibility of individual temperature and climate control as a function of the current body temperature for the person wearing a piece of clothing designed according to the present invention, so that persons subject especially to thermal stress and at the same time also to physical stress, for example, firemen, pilots or soldiers, benefit from the present invention.

The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

FIG. 1 is a cross sectional view through a vest for body climate control;

FIG. 2 is a schematic view of a vest for body climate control;

FIG. 3 is a schematic view of a device for body climate control, and

FIG. 4 is a flow chart of the process for body climate control.

### DESCRIPTION OF THE PREFERRED EMBODIMENTS

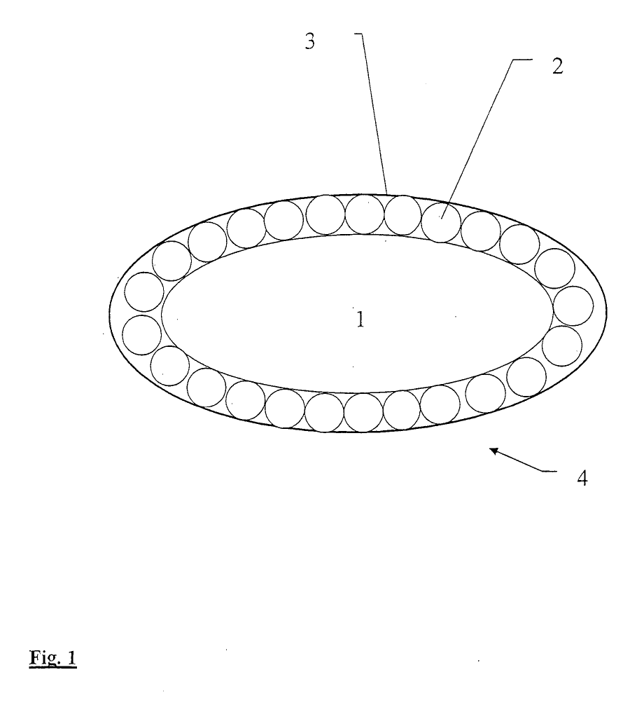

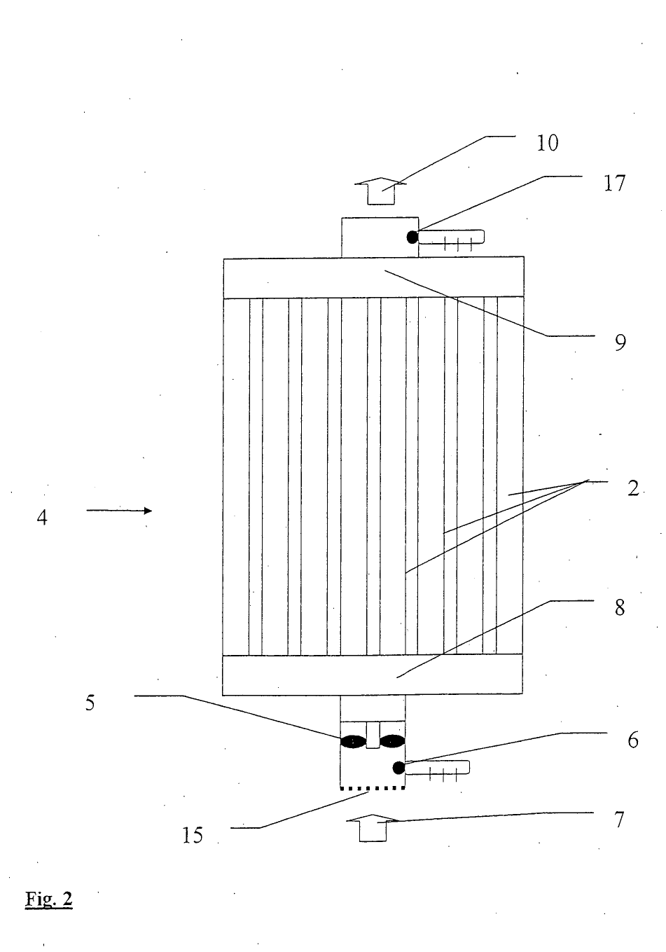

A device for body climate control has, according to FIG. 1, a piece of clothing, which is designed, for example, as a vest 4 and contains ventilation channels 2, to which ambient air 7 (FIG. 2) can be admitted via its inlet. The ventilation channels 2 are preferably permeable to water vapor and absorb moisture and heat from the skin of the user 1 via their walls. The ambient air 7, which is sent through the ventilation channels 2, absorbs the moisture by diffusion and transports same via the outlet of the ventilation channels 2 into the environment. For screening against high external heat radiation and high ambient temperature, the vest 4 may be closed toward the outside by a heat protection textile 3 made of, e.g., Nomex.

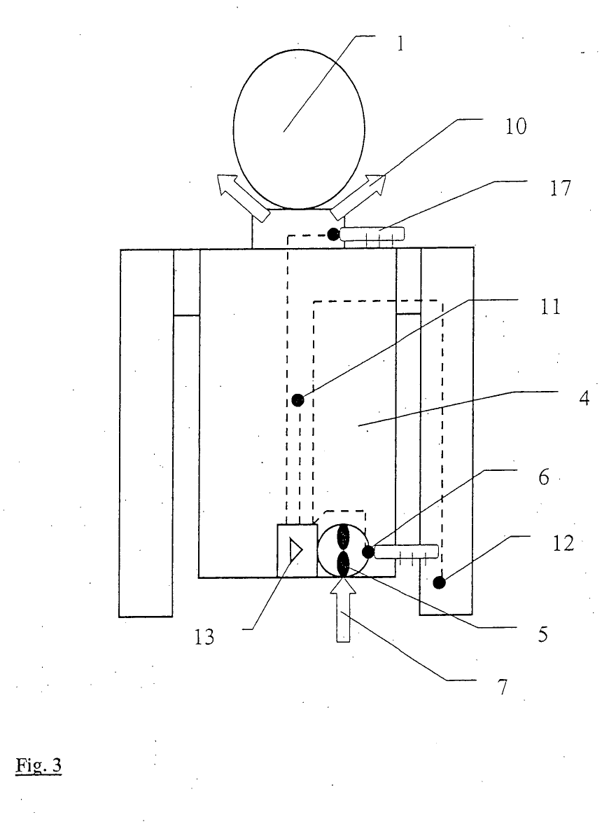

FIG. 2 shows the vest 4 provided with, e.g., parallel, especially equidistant ventilation channels 2, with additional components. A blower 5 draws in ambient air 7 and presses same into the ventilation channels 2 of the vest 4. In a first distributor 8, the ambient air 7 drawn in is distributed among the individual ventilation channels 2. The outlet of the ventilation channels 2 leads into a second distributor 9 which leads to the device outlet 10, which leads to the environment. A temperature sensor for the ambient temperature 6 is arranged upstream of the first distributor 8 and the blower 5 in the example. A particle filter 15, which is optionally arranged upstream, shall protect the user 1 from hazardous substances. In addition, a gas filter may be provided in order to optionally protect the user 1 from toxic gases. A temperature sensor 17 for determining the outlet temperature is optionally located in the outlet area of the ventilation channels 2. A device for body climate control is schematically shown in FIG. 3 with the vest 4, the blower 5, the temperature sensor and optional humidity sensor for the ambient temperature 6 and the outlet temperature 7 as well as with the temperature sensor for determining the temperature 11 near the core of the body and with the temperature sensor for determining the peripheral temperature 12 of the body. Finally, the sensors and the blower 5 are connected with the measuring and evaluating unit 13, which controls the device. In a special embodiment, the measuring and evaluating unit 13 is equipped with a communication unit for telemetric signal transmission, e.g., to a central unit, and optionally with a pulse sensor and/or with electrodes for determining the cardiac activity of the user 1. The energy supply is preferably ensured by means of batteries.

The device operates as follows: In case of rising temperature of the user 1, the peripheral temperature of the body rises at first, which is detected with the temperature sensor 12 and is measured, for example, at the wrist. With rising temperature near the core of the body (temperature sensor 11), which is measured, for example, in the chest region, the blood supply of the periphery of the body is improved due to the dilation of the blood vessels, as a result of which the temperature of the periphery of the body increases as well and the difference between the temperature near the core of the body and the peripheral temperature of the body decreases. It can be assumed in this case that the body of the user 1 is subject to great stress and generates a large amount of heat, which he cannot sufficiently release to the environment. Beginning from a temperature of somewhat higher than 37 C near the core of the body, the body begins to sweat. The blower 5 is switched on at the latest when this temperature limit value for the temperature near the core of the body is exceeded in order to deliver cooler and drier ambient air 7 through the vest 4 and thus remove moisture and heat from the body of the user 1. When a limit value for the ambient temperature 6, e.g., 45 C, is exceeded, the measuring and evaluating unit 13 switches off the blower 5 in order to avoid overheating or even burning of the user 1. When an additional humidity sensor for the ambient air 7 is used, this temperature limit value for the ambient air 7 can be set higher if the humidity of the ambient air is low, because the air being transported can absorb additional moisture in the ventilation channels 2. In addition, a heater may be present as an actuator for the device besides the blower 5.

If the difference between the temperature near the core of the body and the peripheral temperature of the body drops below a preset limit value and the temperature near the core of the body exceeds a preset limit value during increasing stress of the user 1, the measuring and evaluating unit 13 increases the cooling output by changing the rate of air delivery, which depends on the speed of the blower 5. If, on the other hand, the temperature near the core of the body is, e.g., below 36.5 C, the measuring and evaluating unit 13 switches off the blower 5.

Instead of the described adaptation to the body temperatures with the two temperature sensors 11, 12, the interior space temperature in the ventilation channels 2 and optionally the humidity in the interior space can be used by means of the temperature sensor for the outlet temperature 17 in a simple arrangement. The outlet temperature 17 and the corresponding humidity, which is measured in the outlet 10 of the ventilation channels 2, increase with increasing body temperature. The delivery output is then raised by increasing the speed of the blower 5. If the outlet temperature 17 is still very low and approximately corresponds to the ambient temperature 6, cooling is not necessary, so that the speed is reduced or the blower 5 is switched off. When a preset temperature limit value is exceeded, the blower 5 is switched on or the speed is increased.

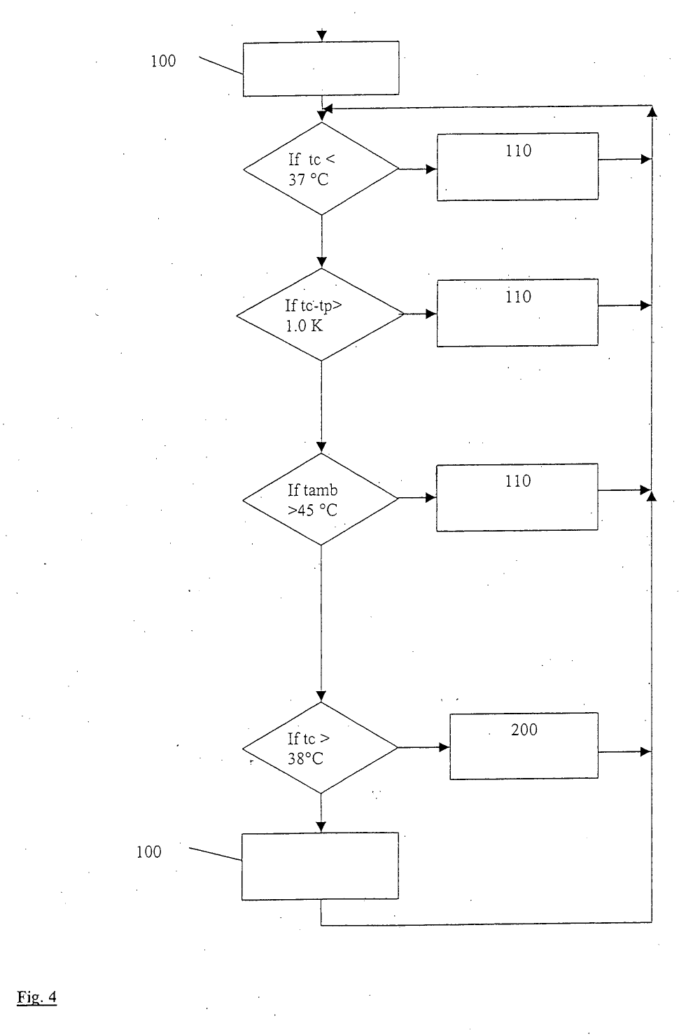

FIG. 4 shows a flow chart for a process for body climate control: The blower 5 is first switched on at a preset speed according to 100. When the condition is met, the blower 5 is either switched off according to 110 or its speed is increased 200 when a preset limit value of the temperature (tc) near the core of the body, e.g., 38 C, is exceeded. The blower 5 is switched off when the measured temperature (tc) near the core of the body drops below a preset limit value of, e.g., 37 C, when the difference between the measured temperature (tc) near the core of the body and the peripheral temperature (tp) of the body (tc-tp) exceeds a preset limit value of, e.g., 1 K or when the ambient temperature 6 (tamb) exceeds a preset limit value of, e.g., 45 C.

While specific embodiments of the invention have been shown and described in detail to illustrate the application of the principles of the invention, it will be understood that the invention may be embodied otherwise without departing from such principles.

1. A process for body climate control with a piece of clothing defining ventilation channels, with a temperature sensor measuring a temperature near a core of a body, with a temperature sensor measuring a peripheral temperature of the body, with a blower ventilating the ventilation channels with ambient air, and with a measuring and evaluating unit connected with the temperature sensors and with the blower, the process comprising the steps of:
switching on the blower with a preset speed;
switching off the blower when the measured temperature near the core of the body is below a lower limit value;
switching off the blower when a difference between the measured temperature near the core of the body and the peripheral temperature of the body exceeds a preset difference limit value, and
adjusting a speed of the blower as a function of one of the measured temperature near the core of the body and the peripheral temperature of the body.

2. A process in accordance with claim 1, wherein:
ambient temperature is measured by a temperature sensor;
said speed of the blower is adjusted as a function of said ambient temperature.

3. A process in accordance with claim 2, wherein:
switching on the blower after one of a lower limit value for said ambient air temperature is exceeded, and after a lower limit value for the temperature near the core of the body is exceeded.

4. A process in accordance with claim 1, wherein:
the measured temperatures and the differences between the temperature near the core of the body and the peripheral temperature of the body are polled one after another in time by said measuring and evaluating unit and compared with stored values for setting said blower.

5. A process in accordance with claim 1, further comprising:
measuring a humidity of the ambient air;
adjusting the blower as a function of the humidity.

6. A process in accordance with claim 5, further comprising:
switching off said blower only after a preset upper limit value for the ambient temperature has been exceeded by a preset amount, said preset amount increases with decreasing humidity.

7. A process in accordance with claim 1, further comprising:
increasing a speed of the blower after a difference between the temperature near the core of the body and the peripheral temperature of the body decreases by a preset amount and a preset limit value for the temperature near the core of the body is exceeded at the same time.

8. A device for body climate control of a user, the device comprising:
a piece of clothing defining ventilation channels having an inlet and an outlet connected to a surrounding environment;
a temperature sensor measuring a body temperature of the user of said piece of clothing;
a blower delivering ambient air into said ventilation channels;
a measuring and evaluating unit connected to said temperature sensor and to said blower.

9. A device in accordance with claim 8, wherein:
said temperature sensor is arranged for measuring a temperature near a core of a body of the user;
another temperature sensor is arranged for measuring a peripheral temperature of the body.

10. A device in accordance with claim 8, wherein:
said measuring and evaluating unit is additionally connected with a temperature sensor for ambient temperature.

11. A device in accordance with claim 8, further comprising:
a humidity sensor measuring humidity at said outlet of said ventilation channels, said humidity sensor being connected to said measuring and evaluating unit.

12. A device in accordance with claim 8, wherein:
said piece of clothing is one of a vest, pants or a complete safety suit.

13. A device in accordance with claim 9, wherein:
one of said temperature sensor for determining the temperature near the core of the body and said temperature sensor for determining the peripheral temperature of the body are incorporated in said piece of clothing.

14. A device in accordance with claim 8, wherein:
said measuring and evaluating unit is provided with a communication unit for telemetric signal transmission.

15. A device in accordance with claim 8, wherein:
said ventilation channels are defined by a material impermeable to water vapor.

16. A device in accordance with claim 11, further comprising:
another humidity sensor for ambient air and connected to said measuring and evaluation unit.

17. A device in accordance with claim 8, wherein:
said temperature sensor measures outlet temperature at said outlet of said ventilation channels.

18. A device in accordance with claim 8, further comprising:
a heater in flow connection with said ventilation channels and delivering heated air to said ventilation channels.

19. A device in accordance with claim 8, further comprising:
one of a pulse sensor and/or electrodes for determining a cardiac activity of said user, said measuring and evaluating unit being connected to said one of said pulse sensor and electrodes.

20. A device in accordance with claim 9, wherein:
said measuring and evaluating unit is additionally connected with a temperature sensor for measuring an ambient temperature;
a humidity sensor measures humidity at said outlet of said ventilation channels, said humidity sensor being connected to said measuring and evaluating unit;
said piece of clothing is one of a vest, pants or a complete safety suit;
one of said temperature sensor for determining the temperature near the core of the body and said temperature sensor for determining the peripheral temperature of the body are incorporated in said piece of clothing;
said measuring and evaluating unit is provided with a communication unit for telemetric signal transmission;
said ventilation channels are defined by a material impermeable to water vapor;
another humidity sensor for measuring ambient air is connected to said measuring and evaluation unit;
still another temperature sensor measures outlet temperature at said outlet of said ventilation channels;
a heater is in flow connection with said ventilation channels and delivers heated air to said ventilation channels;
one of a pulse sensor and/or electrodes for determining a cardiac activity of said user, are connected to said measuring and evaluating unit.

21. A process for body climate control of a user, the process comprising the steps of:
providing a piece of clothing arranged on the user, said clothing defining a plurality of ventilation channels with inlets and outlets in flow connection to a surrounding environment;
measuring a core temperature of the user of said piece of clothing;
measuring a peripheral temperature of the user of said piece of clothing;
providing a blower delivering ambient air into said ventilation channels;
operating said blower;
stopping operation of said blower when said core temperature is below a lower limit;
determining a temperature difference between said core temperature and said peripheral temperature;
stopping operation of said blower when said temperature difference exceeds a difference limit;
adjusting a speed of said blower as a function of said core temperature and said peripheral temperature.
